(19) United States
(12) Patent Application Publication
(10) Pub. No.: US 2009/0111131 A1
(43) Pub. Date: Apr. 30, 2009

(54) Method of detecting protein losing enteropathy in animals

(76) Inventor: Seth Fishman, Boynton Beach, FL (US)

Correspondence Address: MORGAN, LEWIS & BOCKIUS LLP, 1701 MARKET STREET, PHILADELPHIA, PA, 19103-2921, US

(21) Appl. No.: 12/283,653

(22) Filed: Sep. 15, 2008

Related U.S. Application Data
(60) Provisional application No. 60/993,651, filed on Sep. 13, 2007.

(51) Int. Cl. G01N 33/53; G01N 33/68

(52) U.S. Cl. 435/7.92; 436/86; 436/501

(57) ABSTRACT

The present invention provides a method and kit for the detection of Protein Losing Enteropathy (PLE) in animals. The method includes the steps of (a) obtaining a biological sample from an animal to be tested and (b) determining the absence or presence of albumin in the biological sample via a kit comprising an immunoassay utilizing a species-specific anti-albumin antibody.

### STATEMENT OF RELATED APPLICATIONS

This application claims the benefit of U.S. Patent Application No. 60/993,651 filed on Sep. 13, 2007, which is incorporated herein by reference in its entirety.

### BACKGROUND OF THE INVENTION

Protein losing enteropathy refers to any condition of the gastrointestinal tract in humans and animals that results in a net loss of protein from the body. Common causes of protein losing enteropathy include celiac disease, Crohn's disease, short bowel syndrome (where the absorptive area for proteins is decreased), intestinal lymphangiectasia, amyloidosis, enteropathy caused by NSAIDs, and giardiasis. The diagnosis of protein losing enteropathy is typically made by excluding other causes of protein loss, such as nephrotic syndrome. Endoscopy and barium imaging can be used to localize the cause of the protein loss in the bowel. However, these methods are costly, time consuming, and generally only feasible in humans. A need therefore exists for a relatively inexpensive and quick method and kit for the detection of protein losing enteropathy in animals.

### SUMMARY OF THE INVENTION

The present invention relates to a method and kit for the detection of Protein Losing Enteropathy (PLE) in animals. Preferred animals to test for PLE are domesticated animals with horses, cows, dogs, cats, and small reptiles being the most preferred. Method and kit embodiments disclosed herein are based on discovering the absence or presence of albumin in a biological sample from an animal as an indicator of PLE. The most preferred method or kit embodiment is an immunoassay utilizing a species-specific anti-albumin antibody.

### DETAILED DESCRIPTION OF THE INVENTION

The present invention relates to a method and kit for the detection of Protein Losing Enteropathy (PLE) in animals. PLE is the loss of blood serum proteins into fecal matter as a result of compromised epithelial tight junctions in the gastrointestinal tract. Under normal circumstances, tight junctions between epithelial enterocytes in the gastrointestinal tract by virtue of their tight junctions prevent the leakage and or loss of various molecules and proteins from the intravascular space into the gastrointestinal lumen. Various conditions ranging from inflammatory, infectious (bacterial, viral and parasitic), to autoimmune may cause inflammation and or edema, which may then disrupt the tight junctions, resulting in hypoalbuminemia, hypoproteinemia, and severe to possibly fatal fluid retention in various body compartments such as the chest. A major indicator of PLE is the presence or increased concentration of serum albumin in the fecal mater.

A method of the present invention can be generally accomplished by:
(a) obtaining a biological sample from an animal; and
(b) determining the absence or presence of albumin in the biological sample via a kit comprising an immunoassay utilizing a species-specific anti-albumin antibody.

As used herein, the term “biological sample” is meant to encompass any specimen obtained from an animal that can be used to measure albumin leakage through the epithelial tight junctions of the gastrointestinal tract, with fecal matter being the most preferred.

As used herein, the term “animal” is meant to encompass any non-human organism capable of developing PLE. Preferred animals to test for PLE are domesticated animals with horses, cows, dogs, cats, and small reptiles being the most preferred.

As used herein, the term “kit” is meant to encompass any device capable of being contacted by a biological sample and comprising an immunoassay utilizing a species-specific albumin antibody.

As used herein, the term “assay” is meant to encompass any immunoassay designed to use antibodies including, but not limited to: sandwich immunoassay, radioimmunoassay, enzyme-linked immunosorbant assay (ELISA), sandwich enzyme-linked immunosorbant assay, fluorescence immunoassay, competitive exclusion immunoassay, radial diffusion immunoassay, “dipstick” immunoassay, and laminar flow immunoassay. The preferred assay is an immunoassay designed to use antibodies antigenic to the serum albumin of the desired species to detect the presence of serum albumin in a biological sample, most preferably in fecal matter.

As used herein, the term “antibody” is meant to encompass any antibody antigenic to serum album. The antibodies used in the present invention can be polyclonal, monoclonal, or antibody fragments (FAB). The antibodies for the present invention may be prepared in either the classic methods of animal inoculation and isolation from blood serum, formation of cell line hybridomas and establishment of cell cultures producing monoclonal antibodies, or via newly developed recombinant techniques for the rapid screening, isolation, and production of monoclonal antibodies in bacterial cultures. A preferred antibody used in the present invention will be specific for the target species of the diagnostic tool (i.e. equine anti-albumin antibodies will be used in the Equine PLE Diagnostic Indicator Test; bovine anti-albumin antibodies will be used in the Bovine PLE Diagnostic Indicator Test; canine anti-albumin antibodies will be used in the Canine PLE Diagnostic Indicator Test; feline anti-albumin antibodies will be used in the Feline PLE Diagnostic Indicator Test, reptilian anti-albumin antibodies will be used in the Reptilian PLE Diagnostic Indicator Test, etc.).

In one embodiment, the kit is an analytical test device incorporating a porous solid phase material carrying in a first zone a labeled reagent that is retained in the first zone while the porous material is in the dry state but free to migrate through the porous material when the porous material is moistened by the application of the biological sample to the sample pad; the porous material carrying in a second zone, which is spatially distinct from the first zone, an unlabelled specific binding reagent having specificity for albumin and is capable of participating with the labeled reagent in either a “sandwich” or a “competition” reaction, the unlabelled specific binding reagent being firmly immobilized on the porous material such that it is not free to migrate when the porous material is in the moist state. The kit will include instructions on the method of: (a) obtaining a biological sample from an animal to be tested and (b) determining the absence or presence of albumin in the biological sample via the included immunoassay.

In one embodiment, the porous solid phase material is a nitrocellulose membrane.

In one embodiment, the labeled reagent is a species-specific anti-albumin antibody.

In one embodiment, the assay will be a laminar flow immunoassay (LFIA) that utilizes antibodies specific for albumin to detect the presence or absence of such in the biological sample.

In one embodiment, the assay will be considered a “sandwich” assay as described by David et al. (U.S. Pat. No. 4,376,110), herein incorporated in its entirety. The preferred form of this assay has been generally described by May et al. (U.S. Pat. No. 5,602,040), herein incorporated in its entirety.

In one embodiment, the present invention will use species-specific anti-albumin antibodies immobilized on a nitrocellulose membrane as the capture antibody. Additionally, a control line of immobilized antibody specific for other antibodies will be painted on the nitrocellulose membrane after the test line of anti-serum albumin antibodies. A mobile phase, labeled (fluorescent or colormetrically labeled) anti-albumin antibody will be contained in the sample pad. When the biological sample is applied to the sample pad, the sample will interact with the mobile phase, labeled anti-albumin, and will elute across the nitrocellulose membrane. If there is albumin analyte present in the biological sample, the mobile phase, labeled antibodies will bind with the albumin present in the sample, thus attaching a tag to the analyte. The tagged analyte will migrate across the nitrocellulose membrane where it will additionally interact with and bind to the immobilized anti-albumin antibodies, thus forming a line. If no albumin analyte is present in the sample, the tagged anti-serum albumin antibodies in the mobile phase will migrate, unattached to any analyte, across the nitrocellulose membrane, bypassing the test line. In all cases the final control line is specific for the tagged antibodies and will always capture these antibodies, resulting in a control line indicating the test worked as required.

What is claimed is:

1. A kit comprising an assay for determining and diagnosing Protein Losing Enteropathy (PLE) via the detection of key proteins in a biological sample.

2. The assay of claim 1, wherein the biological sample used is fecal matter.

3. The assay of claim 1, wherein the key protein detected is serum albumin.

4. The assay of claim 1, wherein the assay is an immunoassay.

5. The assay of claim 4, wherein the immunoassay is a sandwich immunoassay.

6. The assay of claim 4, wherein the immunoassay is a radioimmunoassay.

7. The assay of claim 4, wherein the immunoassay is an enzyme-linked immunosorbant assay (ELISA).

8. The assay of claim 4, wherein the immunoassay is a sandwich enzyme-linked immunosorbant assay.

9. The assay of claim 4, wherein the immunoassay is a fluorescence assay.

10. The assay of claim 4, wherein the immunoassay is a competitive exclusion assay.

11. The assay of claim 4, wherein the immunoassay is a laminar flow assay.

12. The assay of claim 4 wherein the immunoassay will use antibodies specific for serum albumin.

13. The assay of claim 1, wherein the assay is specific to equines.

14. The assay of claim 1, wherein the assay is specific to bovines.

15. The assay of claim 1, wherein the assay is specific to canines.

16. The assay of claim 1, wherein the assay is specific to felines.

17. The assay of claim 1, wherein the assay is specific to reptilians.

18. A method for the detection of Protein Losing Enteropathy (PLE) in animals.

19. The method of claim 18, wherein the method includes the steps of:
(a) obtaining a biological sample from an animal to be tested; and
(b) determining the absence or presence of albumin in the biological sample via a kit comprising an immunoassay utilizing a species-specific anti-albumin antibody.
